SEQUENCE LISTING (23 sequences)

SEQ ID NO: 1 - DNA, 368 bp, Trichomonas vaginalis
cggtaggtgaacctgccgttggatcagttctagttttaataactaacaccaacttcttttattaaacaaaaaccaatacaaaattaaaaactaacttcatcaaaaaccaagtctctaagcaatggatgtcttggctcctcacacgatgaagaacgtggcataatgtgttaagtaaccggagttgcaaacatcatgacaggttaatctttgaatgcaaattgcgctaaactcgatctcggtcgagaagcatgggtgtgacagtactacatcttttataataattcttattctaagcgaataagtaaataatatataagacaaacaacacgtagtctgccatacgcaggaagacccgctgaactgaagca

SEQ ID NO: 2 - DNA, 161 bp, Artificial Sequence
  Synthetic
gcaatggatgtcttggctcctcacacgatgaagaacgtggcataatgtgttaagtaaccggagttgcaaacatcatgacaggttaatctttgaatgcaaattgcgctaaactcgatctcggtcgagaagcatgggtgtgacagtactacatcttttataat

SEQ ID NO: 3 - DNA, 60 bp, Artificial Sequence
  Synthetic
gagttgcaaacatcatgacaggttaatctttgaatgcaaattgcgctaaactcgatctcg

SEQ ID NO: 4 - DNA, 20 bp, Artificial Sequence
  Synthetic
gcaatggatgtcttggctcc

SEQ ID NO: 5 - DNA, 20 bp, Artificial Sequence
  Synthetic
tggctcctcacacgatgaag

SEQ ID NO: 6 - DNA, 20 bp, Artificial Sequence
  Synthetic
acacgatgaagaacgtggca

SEQ ID NO: 7 - DNA, 20 bp, Artificial Sequence
  Synthetic
ccggagttgcaaacatcatg

SEQ ID NO: 8 - DNA, 23 bp, Artificial Sequence
  Synthetic
tgacaggttaatctttgaatgca

SEQ ID NO: 9 - DNA, 25 bp, Artificial Sequence
  Synthetic
aggttaatctttgaatgcaaattgc

SEQ ID NO: 10 - DNA, 19 bp, Artificial Sequence
  Synthetic
ctaaactcgatctcggtcg

SEQ ID NO: 11 - DNA, 21 bp, Artificial Sequence
  Synthetic
cgagatcgagtttagcgcaat

SEQ ID NO: 12 - DNA, 21 bp, Artificial Sequence
  Synthetic
cgagtttagcgcaatttgcat

SEQ ID NO: 13 - DNA, 25 bp, Artificial Sequence
  Synthetic
gatgtagtactgtcacacccatgct

SEQ ID NO: 14 - DNA, 29 bp, Artificial Sequence
  Synthetic
attataaaagatgtagtactgtcacaccc

SEQ ID NO: 15 - DNA, 24 bp, Artificial Sequence
  Synthetic
ggcagactacgtgttgtttgtctt

SEQ ID NO: 16 - DNA, 20 bp, Artificial Sequence
  Synthetic
caggttaatctttgaatgca

SEQ ID NO: 17 - DNA, 2765 bp, Trichomonas vaginalis
cgtagtctgccatacgcaggaagacccgctgaactgaagcatctcattaagcggaggaaaagaaactaactaggattcccttagtaaaggcgatcgaaaagggaacagcccaacgtcttaatcccgcaagggaaatgtagtcggttcacctattgcactggtctaagtcagatggaatgctgcgcgattcagagtaaaagcctcgtatgaccaaccgcaagcttacgatggaccggggagagtcggatactttgagagtagtgtcttaagggggtggtatgtcccatccaaggctaaataacgtgaggagaccgatagattacaagtagtgtgaacgaaagatgaaaagaactctaaaacagagggtgaaaagttcctgaacttgcttagggttaaggctatttatagtacccgtcttgaaacacggattagggagtttatctgtagtgcgagtctttgcacgttaactgtaaaggcgtagagaaagcaactggcgtttgcatggctactgagcattactgataggacccgaaaggtggtgaactttgcctgcacaggttgaagtcaggggaaactctgatggaggaccgtacgggttattgatgtgcaaatcatttttctgatgtaggtaaaggtgcgaaagactcatcgaaccacctaatagctggtttcctccgaagtttccctcaggatagctgtaatgcatttagttaaggggagtagagatatgggtagaggtgcagggagccttcgggttctcaacttatttccaaacttcgaacctccttgttaggcatttacctaactggaaatgcttaaatgaattagcgttacaagtgggctctgaccggtaagcggtcatggcgaaatgggatgaaccatacgtctgaataaggtgccgaaatgtactatcactgataccataaagggagtccatgtatttagacagcaggtcggtgcccctaaaagcaggtatccgctaaagagtgtgttaaaactcacctgccgaatactagggccccgaaaatgaatggcgcatatagtactaccgatctcagacatcagttctattgaactgagagtagggggatgtggacatttttgtgaaggtgcaatgtgagttggactggaagaatgtctattacagatcctaattgaagtagcgattatccaggcttgtatcctgggggctgatgaggagtagggtttctggacaacttaaacagatccagagttagtcgatcctaatctcaataatgggagaaagggagtacagttgatattctgtaactgacaacagttcgcggcaacgcatactatttttctccgccaatcttataccgttggtagggatctctttcctacatgacagtactaatacccagggaaactggtcatcaggcgaactgcggcgttctgctggcaaaacatagcttcggttatgtcaatggctattggattggtccatgaagcgaaaaatagtttactgtgtcactcgtaccgataaccgcatcaggtcttctaggtaagtagcctatggccattgaggcatgatagttcagggaattcggccaattagagctgtaacttcgggataaagcttggctctgatgaaaatcagaaccgattgttacgcggagagcccaactgtttaataaaaacgtagtgtcgcgctgtggtagtatagccagtgaacgcgacatgaatcctgctcagtgctgtgtctgttaatatggttcaagccaacgaagcaccggtaaatagctggggtaactatgactcttatcaaagtagcgaaatacctcgtcacttaattagtgacctgcatgaacggagctatgaggttctcactgtcccaaactgtctctcagcgaaccaactgtagtgggaacggtcactagatatccagcggggaaagaagaccctgtagagcttcactttggtctagtcttgtatagttcctgcgggggtgtagactaggtgggagagcaatcgattctgaaaaaccactaccctgtttggaattatgcttacctatcacaaggaataggactagatagaaagtttggctggggcggcacaccttcgaaagagcagcgaaggtgtcctaagttgagctcagtgaggacagaaatctcacgttgaggataaggccaaaagctcaatttactgtgcctttttgtactaggatcagttacgaaagtaaggcctaacgatccctaaatagcctcaggtgcgtgaggattggggtgacagacaagttacctccgggacaactggcttgtggctgctaagagatcaaatcaacgcagctttttgattcttcgatgtcggctcttcctatcctagtgatgtacaagttgctaagggtcggattgttcacccgtattagggatcgtgagctgggtttagaccgtcgtgagccaggtttgttttatcctactggatatagcatcatgatagtagattgatttagtacgagaggaactgttgattatggcctctggttagcgattatcggaaacggtatgtcgcgcagctacgccgtctgggataatggctgaaagcctcttaagccagaagcccatctagaaagtgatgcacgtgatgttggaccgatacgaataatcatactcaaaaggtccgcttcataggtaacccaatgtagaagacattgtgtgaacgaaggtactgtaagcagtggagtggcattacgtccacgacctgttgaggttcagcttcgcttctaggtttccaaatatttt

SEQ ID NO: 18 - DNA, 119 bp, Artificial Sequence
  Synthetic
cttcataggtaacccaatgtagaagacattgtgtgaacgaaggtactgtaagcagtggagtggcattacgtccacgacctgttgaggttcagcttcgcttctaggtttccaaatatttt

SEQ ID NO: 19 - DNA, 90 bp, Artificial Sequence
  Synthetic
aggtaacccaatgtagaagacattgtgtgaacgaaggtactgtaagcagtggagtggcattacgtccacgacctgttgaggttcagcttc

SEQ ID NO: 20 - DNA, 27 bp, Artificial Sequence
  Synthetic
aggtaacccaatgtagaagacattgtg

SEQ ID NO: 21 - DNA, 22 bp, Artificial Sequence
  Synthetic
gaagctgaacctcaacaggtcg

SEQ ID NO: 22 - DNA, 19 bp, Artificial Sequence
  Synthetic
ggtactgtaagcagtggag

SEQ ID NO: 23 - DNA, 368 bp, Trichomonas tenax
cggtaggtgaacctgccgttggatcagttctagtttaataactaataccaacttctttttattaatcaaaaactaatacaattataaataactaacttcatcaaaaatcaagtctctaagcaatggatgtcttggctcctcacacgatgaagaacgtggcataatgtgttaagtaaccggagttgcatacatcatgacaggttaatctttgaatgcaaattgcgctaaacttggcttcggctgagaagcatgcgtgtaacagtacaacataatttataataattcttattctaagcgaataagtaaataaattataagacaaacttacgttgtctgctatacgcaggaagacccgctgaactgaagca

(19) United States
(12) Patent Application Publication
(10) Pub. No.: US 2012/0141992 A1
(43) Pub. Date: Jun. 7, 2012

(54) COMPOSITIONS AND METHODS FOR TRICHOMONAS VAGINALIS DIAGNOSTIC TESTING

(75) Inventors: Mitra Choudhury Singhal, Edmonds, WA (US); Cori Anne Barfield, Seattle, WA (US); Kathryn Watts Weaver, Washington, DC (US)

(73) Assignee: PROGRAM FOR APPROPRIATE TECHNOLOGY IN HEALTH, Seattle, WA (US)

(21) Appl. No.: 13/309,327

(22) Filed: Dec. 1, 2011

Related U.S. Application Data
(60) Provisional application No. 61/418,808, filed on Dec. 1, 2010.

(51) Int. Cl. C12Q 1/68; C07H 21/04

(52) U.S. Cl. 435/6.11; 536/24.33

(57) ABSTRACT

The invention provides methods, reagents and kits for determining the presence of Trichomonas vaginalis in a test sample.

### CROSS-REFERENCE TO RELATED APPLICATION

This application claims the benefit of U.S. Provisional Application No. 61/418,808, filed Dec. 1, 2010.

### STATEMENT OF GOVERNMENT LICENSE RIGHTS

This invention was made with Government support under Grant Number U01 AI070801-05 awarded by the National Institutes of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 38264_SEQ_FINAL_{—}2011-12-01.txt. The text file is 10 KB; was created on Dec. 1, 2011; and is being submitted via EFS-Web with the filing of the specification.

### BACKGROUND

Trichomonas vaginalis (TV) is thought to be the most common parasitic sexually transmitted infection (STI) worldwide, with an estimated incidence of 8 million new cases annually. Infection with TV increases indices associated with enhanced likelihood of women transmitting or acquiring HIV. For example, infection with TV results in an increase in the quantity of detectable cervical HIV, a trend which is reversed with successful antitrichomonal therapy. Proper diagnosis and treatment of the TV, particularly in areas of the world with a high burden of HIV infection, could markedly reduce a woman's likelihood of transmitting or acquiring HIV.

The conventional approach for detecting infection with TV is to perform direct microscopic examination of wet-mounted vaginal or urethral samples in order to observe motile parasites. Although direct microscopic examination is very specific, this approach suffers from poor sensitivity (40-60%) (Wendel et al., Clin. Inf. Dis., 35:576-80, 2002). An alternative diagnostic approach relies on cultivation of TV. Cultivation increases diagnostic sensitivity but has a long turnaround time and thus is not suitable for point-of-care use. PCR assays for TV have been developed that offer assay sensitivities and specificities that are unrivaled by any other diagnostic method.

Accurate and rapid diagnosis of TV at the point of care remains elusive for most women worldwide. Syndromic diagnosis of cervico-vaginal discharge fails to accurately differentiate between cervical and vaginal infections and, indeed, from normal physiologic discharge. TV diagnosis requires user expertise along with a relatively long turnaround time between sample collection and results for reliable and accurate results. Thus, relatively few women with TV are actually detected and treated.

Given the human health implications of TV and the relative inability of existing clinical laboratory methods to selectively and sensitively detect TV from a test sample, a need exists for a sensitive and specific assay which can be used to determine the presence of TV in sample of biological material.

### SUMMARY

In one aspect, the invention provides a method for determining the presence of TV in a test sample. The method according to this aspect of the invention comprises (a) contacting a test sample with a composition comprising at least one primer pair comprising a forward and reverse primer capable of hybridizing to a target region of TV 28S consisting of SEQ ID NO:18 to form a reaction mixture; and (b) subjecting said reaction mixture to amplification conditions suitable to amplify at least a portion of said target region.

In another aspect, the invention provides a set of oligonucleotides for use in amplifying a target region of nucleic acid derived from TV 28S, the set of oligonucleotides comprising a forward and reverse primer, each primer having a target binding region up to 30 nucleotides in length which contains at least 10 contiguous nucleotides which are perfectly complementary to an at least 10 contiguous nucleotide region present in a target sequence consisting of SEQ ID NO:19.

In another aspect, the invention provides an oligonucleotide for use in amplifying a target region of nucleic acid derived from TV, said oligonucleotide having a target binding region of up to 30 bases in length which stably hybridizes to a target sequence selected from the group consisting of SEQ ID NO:18 and SEQ ID NO:19.

In another aspect, the invention provides a kit for determining the presence of TV in a test sample. In accordance with this aspect of the invention, the kit comprises (a) at least one oligonucleotide comprising a target binding region sequence selected from the group consisting of SEQ ID NO:20, SEQ ID NO:21 and SEQ ID NO:22; (b) amplification reagents; and (c) written instructions describing amplification conditions suitable to distinguish between the presence of TV and Trichomonas tenax in the test sample.

The invention thus provides methods, reagents and kits for determining the presence of Trichomonas vaginalis in a test sample.

### SEQUENCE LISTING

SEQ ID NO:1: T. vaginalis U86613 5.8S rRNA full length

SEQ ID NO:2: T. vaginalis target subregion #1: (119 to 279 of SEQ ID NO:1)

SEQ ID NO:3: T. vaginalis target subregion #2 (179 to 238 of SEQ ID NO:1)

SEQ ID NOS:4-16 are primers and probes for 5.8S assay

SEQ ID NO:17: T. vaginalis 28S rRNA full length

SEQ ID NO:18: Target subregion #1 (nt 2647 to 2765 of SEQ ID NO:17)

SEQ ID NO:19: Target subregion #2 (nt 2653-2742 of SEQ ID NO:17)

SEQ ID NO:20: 28S forward (206)

SEQ ID NO:21: 28S reverse (207)

SEQ ID NO:22: 28S probe

SEQ ID NO:23: T. tenax U86615 full length (Genbank Ref. U86615)

### DETAILED DESCRIPTION

Unless specifically defined herein, all terms used herein have the same meaning as they would to one skilled in the art of the present invention. Practitioners are particularly directed to Sambrook et al. (1989) Molecular Cloning: A Laboratory Manual, 2d ed., Cold Spring Harbor Press, Plainsview, N.Y.; and Ausubel et al., Current Protocols in Molecular Biology, John Wiley & Sons, New York (1999) for definitions and terms of art.

The following definitions are provided in order to provide clarity with respect to the terms as they are used in the specification and claims to describe the present invention.

The term “specifically hybridize” as used herein refers to the ability of a nucleic acid to bind detectably and specifically to a second nucleic acid. Polynucleotides specifically hybridize with target nucleic acid strands under hybridization and wash conditions that minimize appreciable amounts of detectable binding to non-specific nucleic acids. Stringent conditions that can be used to achieve specific hybridization are known in the art.

A “target sequence” or “target nucleic acid sequence” as used herein means a nucleic acid sequence of TV, such as a target region of TV 5.8S (e.g., SEQ ID NO:2 or SEQ ID NO:3), or complement thereof, or a target region of TV 28S (e.g., SEQ ID NO:18 or SEQ ID NO:19) that is amplified, detected, or both amplified and detected using one or more of the oligonucleotides primers provided herein. Additionally, while the term “target sequence” sometimes refers to a double stranded nucleic acid sequence, those skilled in the art will recognize that the target sequence can also be single stranded. In cases where the target is double stranded, polynucleotide primer sequences of the present invention preferably will amplify both strands of the target sequence. As described in Examples 1-4, the primer sequences of the present invention are selected for their ability to specifically hybridize with a range of different TV strains and to not hybridize to a near neighbor organism, T. tenax (SEQ ID NO:23).

The term “test sample” as used herein, refers to a sample taken from a subject or other source that is suspected of containing or potentially contains a TV target sequence. The test sample can be taken from any biological source, such as for example, tissue, blood, saliva, sputa, mucus, sweat, urine, urethral swabs, cervical swabs, urogenital or anal swabs, conjunctival swabs, ocular lens fluid, cerebral spinal fluid, milk, ascites fluid, synovial fluid, peritoneal fluid, amniotic fluid, fermentation broths, cell cultures, chemical reaction mixtures and the like. The test sample can be used (i) directly as obtained from the source; or (ii) following a pre-treatment to modify the character of the sample. Thus, the test sample can be pre-treated prior to use by, for example, preparing plasma or serum from blood, disrupting cells or viral particles, preparing liquids from solid materials, diluting viscous fluids, filtering liquids, concentrating liquids, inactivating interfering components, adding reagents, purifying nucleic acids, and the like.

The term “label” as used herein means a molecule or moiety having a property or characteristic which is capable of detection and, optionally, of quantitation. A label can be directly detectable, as with, for example (and without limitation), radioisotopes, fluorophores, chemiluminophores, enzymes, colloidal particles, fluorescent microparticles and the like; or a label may be indirectly detectable, as with, for example, specific binding members. It will be understood that directly detectable labels may require additional components such as, for example, substrates, triggering reagents, quenching moieties, light, and the like to enable detection and/or quantitation of the label. When indirectly detectable labels are used, they are typically used in combination with a “conjugate.” A conjugate is typically a specific binding member that has been attached or coupled to a directly detectable label. Coupling chemistries for synthesizing a conjugate are well known in the art and can include, for example, any chemical means and/or physical means that does not destroy the specific binding property of the specific binding member or the detectable property of the label. As used herein, “specific binding member” means a member of a binding pair, i.e., two different molecules where one of the molecules through, for example, chemical or physical means specifically binds to the other molecule. In addition to antigen and antibody specific binding pairs, other specific binding pairs include, but are not intended to be limited to, avidin and biotin; haptens and antibodies specific for haptens; complementary nucleotide sequences; enzyme cofactors or substrates and enzymes; and the like.

A polynucleotide, in the context of the present invention, is a nucleic acid polymer of ribonucleic acid (RNA), deoxyribonucleic acid (DNA), modified RNA or DNA, or RNA or DNA mimetics (such as, without limitation, PNAs), and derivatives thereof, and homologues thereof. Thus, polynucleotides include polymers composed of naturally occurring nucleobases, sugars and covalent internucleoside (backbone) linkages as well as polymers having non-naturally-occurring portions that function similarly. Such modified or substituted nucleic acid polymers are well known in the art and, for the purposes of the present invention, are referred to as “analogues.” For ease of preparation and familiarity to the skilled artisan, polynucleotides are preferably modified or unmodified polymers of deoxyribonucleic acid or ribonucleic acid.

As used herein, the term “primer” means a polynucleotide which can serve to initiate a nucleic acid chain extension reaction. Typically, primers have a length of 5 to about 50 nucleotides, although primers can be longer than 50 nucleotides.

As used herein, the term “sequence identity” or “percent identical” as applied to nucleic acid molecules is the percentage of nucleic acid residues in a candidate nucleic acid molecule sequence that are identical with a subject nucleic acid molecule sequence (such as the nucleic acid molecule sequence set forth in SEQ ID NO:2), after aligning the sequences to achieve the maximum percent identity, and not considering any nucleic acid residue substitutions as part of the sequence identity. No gaps are introduced into the candidate nucleic acid sequence in order to achieve the best alignment. Nucleic acid sequence identity can be determined in the following manner. The subject polynucleotide molecule sequence is used to search a nucleic acid sequence database, such as the Genbank database, using the program BLASTN version 2.1 (based on Altschul et al., Nucleic Acids Research 25:3389-3402 (1997)). The program is used in the ungapped mode. Default filtering is used to remove sequence homologies due to regions of low complexity as defined in Wootton, J. C., and S. Federhen, Methods in Enzymology 266:554-571 (1996). The default parameters of BLASTN are utilized.

The present invention further encompasses homologues of the polynucleotides (i.e., primers and detection probes) having nucleic acid sequences set forth in SEQ ID NOS:4-16 and 20-22). As used herein, the term “homologues” refers to nucleic acids having one or more alterations in the primary sequence set forth in any one of SEQ ID NOS:4-16 and 20-22, that does not destroy the ability of the polynucleotide to specifically hybridize with a target sequence, as described above. Accordingly, a primary sequence can be altered, for example, by the insertion, addition, deletion or substitution of one or more of the nucleotides of, for example, SEQ ID NOS:4-16 and 20-22. Thus, in one embodiment, homologues have a length in the range of from 10 to 30 nucleotides and have a consecutive sequence of at least 10, 11, 12, 13, 14, 15, 16, 17, 18, 19, 20, 21, 22, 23, 24, 25 or more nucleotides of the nucleic acid sequences of SEQ ID NO:4-16 and 20-22, and retain the ability to specifically hybridize with a target sequence, as described above. Ordinarily, the homologues will have a nucleic acid sequence having at least 85%, 90%, or 95% nucleic acid sequence identity with a nucleic acid sequence set forth in SEQ ID NOS:4-16 and 20-22. In some embodiments, homologues have a length in the range of from 10 to 30 nucleotides and have a nucleotide sequence substantially identical to a nucleotide sequence set forth as SEQ ID NOS:4-16 and 20-22, with the difference being the presence of 1, 2 or 3 mismatches, provided that the homologues do not contain two or more consecutive mismatches.

The polynucleotides of the present invention thus comprise primers and probes that specifically hybridize to a target sequence of the invention, for example the nucleic acid molecules having any one of the nucleic acid sequences set forth in SEQ ID NOS:4-16 and 20-22, including analogues and/or derivatives of said nucleic acid sequences, and homologues thereof, that can specifically hybridize with a target sequence of the invention. As described below, polynucleotides of the invention can be used as primers and/or probes to amplify or detect TV.

The polynucleotides according to the present invention can be prepared by conventional techniques well known to those skilled in the art. For example, the polynucleotides can be prepared using conventional solid-phase synthesis using commercially available equipment, such as that available from Applied Biosystems USA Inc. (Foster City, Calif.), DuPont, (Wilmington, Del.), or Milligen (Bedford, Mass.). Modified polynucleotides, such as phosphorothioates and alkylated derivatives, can also be readily prepared by similar methods known in the art. See, for example, U.S. Pat. Nos. 5,464,746; 5,424,414; and 4,948,882.

The polynucleotides according to the present invention can be employed directly as probes for the detection, or quantitation, or both, of TV nucleic acids in a test sample.

In one aspect, the methods comprise detecting the presence of a target region of TV 5.8S in a test sample. The full length nucleotide sequence of TV 5.8S rRNA from the reference TV sequence (Genbank Ref. No. U86613) is set forth as SEQ ID NO:1. In one embodiment, the target region consists of SEQ ID NO:2 (nucleotides 119 to 279 of SEQ ID NO:1). In another embodiment, the target region consists of SEQ ID NO:3 (nucleotides 179 to 238 of SEQ ID NO:1). In accordance with one embodiment of the invention, the method comprises contacting a test sample with a composition comprising at least one primer pair comprising a forward and reverse primer capable of hybridizing to a target region of TV 5.8S consisting of SEQ ID NO:2 (or subregion SEQ ID NO:3) to form a reaction mixture and subjecting said reaction mixture to amplification conditions suitable to amplify a portion of the target region. The amplification conditions are suitable to allow hybridization between the target sequence and the primer pair. In one embodiment, the composition comprises a primer having a target binding region consisting of SEQ ID NO:7. In one embodiment, the composition comprises a primer having a target binding region consisting of SEQ ID NO:11. In some embodiments, the amplified portion of the target region is then detected by a probe that hybridizes to the amplified target region using methods well-known in the art. In one embodiment, the probe comprises a target binding region consisting of SEQ ID NO:16.

In another aspect, the methods comprise detecting the presence of a target region of TV 28S in a test sample. The full length nucleotide sequence of TV 28S rRNA from the reference TV sequence (Genbank Ref. No. AF202181) is set forth as SEQ ID NO:17. In one embodiment, the target region consists of SEQ ID NO:18 (nucleotides 2647 to 2765 of SEQ ID NO:17). In another embodiment, the target region consists of SEQ ID NO:19 (nucleotides 2653 to 2742 of SEQ ID NO:17). In accordance with one embodiment of the invention, the method comprises contacting a test sample with a composition comprising at least one primer pair comprising a forward and reverse primer capable of hybridizing to a target region of TV 28S consisting of SEQ ID NO:18 (or subregion SEQ ID NO:19) to form a reaction mixture and subjecting said reaction mixture to amplification conditions suitable to amplify a portion of the target region. The amplification conditions are suitable to allow hybridization between the target sequence and the primer pair. In one embodiment, the composition comprises a primer having a target binding region consisting of SEQ ID NO:20. In one embodiment, the composition comprises a primer having a target binding region consisting of SEQ ID NO:21. In some embodiments, the amplified portion of the target region is then detected by a probe that hybridizes to the amplified target region using methods well-known in the art. In one embodiment, the probe comprises a target binding region consisting of SEQ ID NO:22.

The polynucleotides (i.e., primers and probes) of the present invention may incorporate one or more detectable labels. Detectable labels are molecules or moieties having a property or characteristic that can be detected directly or indirectly and are chosen such that the ability of the polynucleotide to hybridize with its target sequence is not adversely affected. Methods of labeling nucleic acid sequences are well known in the art (see, for example, Ausubel et al. (1997 & updates), Current Protocols in Molecular Biology, Wiley & Sons, New York).

Amplification procedures are well-known in the art and include, but are not limited to, polymerase chain reaction (PCR), TMA, rolling circle amplification, nucleic acid sequence based amplification (NASBA), and strand displacement amplification (SDA). One skilled in the art will understand that for use in certain amplification techniques the primers may need to be modified, for example, for SDA the primer comprises additional nucleotides near its 5′ end that constitute a recognition site for a restriction endonuclease. Similarly, for NASBA the primer comprises additional nucleotides near the 5′ end that constitute an RNA polymerase promoter. Polynucleotides thus modified are considered to be within the scope of the present invention.

As described in Examples 1 and 2 herein, certain criteria are taken into consideration when selecting the primers and probes for use in the methods of the invention. For example, for primer pairs for use in the amplification reactions, the primers are selected such that the likelihood of forming 3′ duplexes is minimized, and such that the melting temperatures (Tin) are sufficiently similar to optimize annealing to the target sequence and minimize the amount of non-specific annealing. In this context, the polynucleotides according to the present invention are provided in combinations that can be used as primers in amplification reactions to specifically amplify target nucleic acid sequences.

The amplification method of the present invention generally comprises (a) forming a reaction mixture comprising nucleic acid amplification reagents, at least one set of primers of the present invention, and a test sample suspected of containing at least one target sequence; and (b) subjecting the mixture to amplification conditions to generate at least one copy of a nucleic acid sequence complementary to the target sequence.

Step (b) of the above methods can be repeated any suitable number of times (prior to detection of the amplified region), e.g., by thermal cycling the reaction mixture between 10 and 100 times, typically between about 20 and about 60 times, more typically between about 25 and about 45 times, such as between about 30 and 40 times.

Nucleic acid amplification reagents include reagents which are well known and may include, but are not limited to, an enzyme having at least polymerase activity, enzyme cofactors such as magnesium or manganese; salts; nicotinamide adenine dinucleotide (NAD); and deoxynucleotide triphosphates (dNTPs) such as for example deoxyadenine triphosphate, deoxyguanine triphosphate, deoxycytosine triphosphate and deoxythymine triphosphate.

Amplification conditions are conditions that generally promote annealing and extension of one or more nucleic acid sequences. It is well known that such annealing is dependent in a rather predictable manner on several parameters, including temperature, ionic strength, sequence length, complementarity, and G:C content of the sequences. For example, lowering the temperature in the environment of complementary nucleic acid sequences promotes annealing. For any given set of sequences, melt temperature, or Tm, can be estimated by any of several known methods. Typically, diagnostic applications utilize hybridization temperatures that are about 10° C. (e.g., 2° C. to 18° C.) below the melt temperature. Ionic strength or “salt” concentration also impacts the melt temperature, since small cations tend to stabilize the formation of duplexes by negating the negative charge on the phosphodiester backbone. Typical salt concentrations depend on the nature and valency of the cation but are readily understood by those skilled in the art. Similarly, high G:C content and increased sequence length are also known to stabilize duplex formation because G:C pairings involve 3 hydrogen bonds where A:T pairs have just two, and because longer sequences have more hydrogen bonds holding the sequences together. Thus, a high G:C content and longer sequence lengths impact the hybridization conditions by elevating the melt temperature.

Specific amplicons produced by amplification of target nucleic acid sequences using the polynucleotides of the present invention, as described above, can be detected by a variety of methods known in the art. For example, one or more of the primers used in the amplification reactions may be labeled such that an amplicon can be directly detected by conventional techniques subsequent to the amplification reaction. Alternatively, a probe consisting of a labeled version of one of the primers used in the amplification reaction, or a third polynucleotide distinct from the primer sequences that has been labeled and is complementary to a region of the amplified sequence, can be added after the amplification reaction is complete. The mixture is then submitted to appropriate hybridization and wash conditions and the label is detected by conventional methods.

The amplification product produced as above can be detected during or subsequently to the amplification of the target sequence. Methods for detecting the amplification of a target sequence during amplification are outlined above, and described, for example, in U.S. Pat. No. 5,210,015. Gel electrophoresis can be employed to detect the products of an amplification reaction after its completion. Alternatively, amplification products are hybridized to probes, then separated from other reaction components and detected using microparticles and labeled probes.

It will be readily appreciated that a procedure that allows both amplification and detection of target nucleic acid sequences to take place concurrently in a single unopened reaction vessel would be advantageous. Such a procedure would avoid the risk of “carry-over” contamination in the post-amplification processing steps, and would also facilitate high-throughput screening or assays and the adaptation of the procedure to automation. Furthermore, this type of procedure allows “real-time” monitoring of the amplification reaction, as well as more conventional “end-point” monitoring.

The present invention thus includes the use of the polynucleotides in a method to specifically amplify and detect target nucleic acid sequences in a test sample in a single tube format. This may be achieved, for example, by including in the reaction vessel an intercalating dye such as SYBR Green or an antibody that specifically detects the amplified nucleic acid sequence. Alternatively, a third polynucleotide distinct from the primer sequences, which is complementary to a region of the amplified sequence, may be included in the reaction, as when a primer/probe set of the invention is used.

For use in an assay as described above, in which both amplification with polynucleotide primers and detection of target sequences using a polynucleotide probe occur concurrently in a single unopened reaction vessel, the polynucleotide probe preferably possesses certain properties. For example, since the probe will be present during the amplification reaction, it should not interfere with the progress of this reaction and should also be stable under the reaction conditions. In addition, for real-time monitoring of reactions, the probe should be capable of binding its target sequence under the conditions of the amplification reaction and to emit a signal only upon binding this target sequence. Examples of probe molecules that are particularly well-suited to this type of procedure include molecular beacon probes and probes comprising a fluorophore covalently attached to the 5′ end of the probe and a quencher at the 3′ end (e.g., TaqMan® probes).

The present invention, therefore, contemplates the use of the polynucleotides as TaqMan® probes. As is known in the art, TaqMan® probes are dual-labeled fluorogenic nucleic acid probes composed of a polynucleotide complementary to the target sequence that is labeled at the 5′ terminus with a fluorophore and at the 3′ terminus with a quencher. TaqMan® probes are typically used as real-time probes in amplification reactions. In the free probe, the close proximity of the fluorophore and the quencher ensures that the fluorophore is internally quenched. During the extension phase of the amplification reaction, the probe is cleaved by the 5′ nuclease activity of the polymerase and the fluorophore is released. The released fluorophore can then fluoresce and thus produces a detectable signal.

Suitable fluorophores and quenchers for use with the polynucleotides of the present invention can be readily determined by one skilled in the art (see also Tyagi et al., Nature Biotechnol., 16:49-53 (1998); Marras et al., Genet. Anal. Biomolec. Eng., 14:151-156 (1999)). Many fluorophores and quenchers are available commercially, for example from Molecular Probes (Eugene, Oreg.) or Biosearch Technologies, Inc. (Novato, Calif.). Examples of fluorophores that can be used in the present invention include, but are not limited to, fluorescein and fluorescein derivatives such as carboxy fluorescein (FAM®), a dihalo-(C1 to C8)dialkoxycarboxyfluorescein, 5-(2′-aminoethyl)aminonaphthalene-1-sulphonic acid (EDANS), coumarin and coumarin derivatives, Lucifer yellow, Texas red, tetramethylrhodamine, tetrachloro-6-carboxyfluoroscein, 5-carboxyrhodamine, cyanine dyes and the like. Quenchers include, but are not limited to, DABCYL, 4′-(4-dimethylaminophenylazo)benzoic acid (DABSYL), 4-dimethylaminophenylazophenyl-4-dimethylaminophenylazophenyl-4′-maleimide (DABMI), tetramethylrhodamine, carboxytetramethylrhodamine (TAMRA), dihydrocyclopyrroloindole tripeptide minor groover binder (MGB®) dyes and the like. Methods of coupling fluorophores and quenchers to nucleic acids are well known in the art. The present invention thus includes the use of the polynucleotides in a method to specifically amplify and detect target nucleic acid sequences in a test sample in a single tube format. This may be achieved, for example, by including in the reaction vessel an intercalating dye such as SYBR Green or an antibody that specifically detects the amplified nucleic acid sequence. Alternatively a third polynucleotide distinct from the primer sequences, which is complementary to a region of the amplified sequence, may be included in the reaction, as when a primer/probe set of the invention is used.

In accordance with the present invention, therefore, the combinations of two primers and at least one probe, as described above, can be used in either end-point amplification and detection assays, in which the strength of the detectable signal is measured at the conclusion of the amplification reaction, or in real-time amplification and detection assays, in which the strength of the detectable signal is monitored throughout the course of the amplification reaction.

The polynucleotides according to the present invention can also be used in assays to detect the presence and/or quantitate the amount of TV nucleic acid present in a test sample. Thus, the polynucleotides according to the present invention can be used in a method to specifically amplify, detect and quantitate target nucleic acid sequences in a test sample, which generally comprises the steps of (a) forming a reaction mixture comprising nucleic acid amplification reagents, at least one polynucleotide probe sequence that incorporates a label which produces a detectable signal upon hybridization of the probe to its target sequence, at least one polynucleotide primer and a test sample that contains one or more target nucleic acid sequences; (b) subjecting the mixture to amplification conditions to generate at least one copy of the target nucleic acid sequence, or a nucleic acid sequence complementary to the target sequence; (c) hybridizing the probe to the target nucleic acid sequence or the nucleic acid sequence complementary to the target sequence, so as to form a probe:target hybrid; (d) detecting the probe:target hybrid by detecting the signal produced by the hybridized labeled probe; and (e) comparing the amount of the signal produced to a standard as an indication of the amount of target nucleic acid sequence present in the test sample.

One skilled in the art will understand that, as outlined above, step (b) of the above method can be repeated several times prior to step (c) by thermal cycling the reaction mixture by standard techniques known in the art.

Various types of standards for quantitative assays are known in the art. For example, the standard can consist of a standard curve compiled by amplification and detection of known quantities of TV nucleic acids under the assay conditions. Alternatively, an internal standard can be included in the reaction. Such internal standards generally comprise a control target nucleic acid sequence and a control polynucleotide probe. The internal standard can optionally further include an additional pair of primers. The primary sequence of these control primers may be unrelated to the polynucleotides of the present invention and specific for the control target nucleic acid sequence.

In another aspect, the invention provides a set of oligonucleotides for use in amplifying a target region of nucleic acid derived from TV 5.8S, the set of oligonucleotides comprising a forward and reverse primer, each primer having a target binding region. In some embodiments, the target binding region is located at the 3′ end of the oligonucleotide. In some embodiments, the target binding region is from 10 to 30 nucleotides in length and contains at least 10 contiguous nucleotides which are perfectly complementary to an at least 10 contiguous nucleotide region present in a target sequence consisting of SEQ ID NO:3. In one embodiment, the forward primer comprises a target binding region consisting of SEQ ID NO:7. In one embodiment, the reverse primer comprises a target binding region consisting of SEQ ID NO:11: In one embodiment, the detection probe comprises a target binding region consisting of SEQ ID NO:16.

In another aspect, the invention provides a set of oligonucleotides for use in amplifying a target region of nucleic acid derived from TV 28S, the set of oligonucleotides comprising a forward and reverse primer, each primer having a target binding region. In some embodiments, the target binding region is located at the 3′ end of the oligonucleotide. In some embodiments, the target binding region is from 10 to 30 nucleotides in length and contains at least 10 contiguous nucleotides which are perfectly complementary to an at least 10 contiguous nucleotide region present in a target sequence consisting of SEQ ID NO:19. In one embodiment, the forward primer comprises a target binding region consisting of SEQ ID NO:20. In one embodiment, the reverse primer comprises a target binding region consisting of SEQ ID NO:21. In one embodiment, the detection probe comprises a target binding region consisting of SEQ ID NO:22.

In another aspect, the invention provides an oligonucleotide for use in amplifying a target region of nucleic acid derived from TV, said oligonucleotide having a target binding region. In some embodiments, the target binding region is located at the 3′ end of the oligonucleotide. In some embodiments, the target binding region is from 10 to 30 bases in length and stably hybridizes to a target sequence selected from the group consisting of SEQ ID NO:2, SEQ ID NO:3, SEQ ID NO:18 and SEQ ID NO:19. In one embodiment, the oligonucleotide comprises a target binding region which contains at least 10 contiguous nucleotides that are perfectly complementary to at least 10 contiguous nucleotides in said target sequence. In one embodiment, the oligonucleotide does not stably hybridize to any nucleic acid sequences derived from T. tenax. In one embodiment, the oligonucleotide does not stably hybridize to SEQ ID NO:23.

In another aspect, the invention provides a kit for determining the presence of TV in a test sample. In accordance with this aspect of the invention, the kit comprises (a) at least one oligonucleotide comprising a target binding region sequence selected from the group consisting of SEQ ID NO:7, SEQ ID NO:11, SEQ ID NO:16, SEQ ID NO:20, SEQ ID NO:21 and SEQ ID NO:22; (b) amplification reagents; and (c) written instructions describing suitable samples, sample preparation and/or amplification conditions.

In one embodiment, kits for the detection of TV nucleic acids may additionally contain a control target nucleic acid and a control polynucleotide probe. Thus, in one embodiment of the present invention, the kits comprise one of the above combinations of polynucleotides comprising at least two primers and at least one probe, together with a control target nucleic acid sequence, which can be amplified by the specified primer pair, and a control polynucleotide probe. The present invention further provides kits that include control primers, which specifically amplify the control target nucleic acid sequence.

The kits can optionally include amplification reagents, reaction components and/or reaction vessels. Typically, at least one sequence bears a label, but detection is possible without this. Thus, one or more of the polynucleotides provided in the kit may have a detectable label incorporated, or the kit may include reagents for labeling the polynucleotides. One or more of the components of the kit may be lyophilized and the kit may further comprise reagents suitable for the reconstitution of the lyophilized components.

The polynucleotides, methods, and kits of the present invention are useful in clinical or research settings for the detection and/or quantitation of TV nucleic acids. Thus, in these settings the polynucleotides can be used in assays to diagnose TV infection in a subject, or to monitor the quantity of a TV target nucleic acid sequence in a subject infected with TV.

The following examples merely illustrate the best mode now contemplated for practicing the invention, but should not be construed to limit the invention.

### EXAMPLE 1

This Example describes the use of Trichomonas vaginalis 5.8S rRNA Gene Target for a Diagnostic PCR Assay.

Rationale: 5.8S rRNA

The 5.8S rRNA target was sequenced from several TV strains by Felleisen et al. (Parasitology 115:111-119, 1997). This study showed that the sequence is highly conserved among different TV strains but that there is also a high degree of homology to a near neighbor organism, T. tenax. A multiple sequence alignment was constructed using several strains of TV and all near neighbor organisms showing cross-reactivity in a BLAST search of the target of the assay design, the 368 base pair 5.8S rRNA from the reference TV sequence Genbank reference No. U86613 (SEQ ID NO:1). This multiple sequence alignment included one strain of T. tenax (Genbank ref. No. U86615) (SEQ ID NO:23) and six strains of P. hominis (Genbank ref. Nos. AF3442741, AF156964, AY245137, AY758392 and AY349187). The alignment showed that there was a small area of sequence difference between TV and T. tenax that was utilized to develop an assay to differentiate between the two organisms.

### Methods:

PCR Assay Design:

The primer-probe designs were based on multiple sequence alignments that were compiled from annotated sequences in GENBANK and EMBL, as described above. These alignments included sequences from different strains (where available) and also sequences from near neighbor organisms.

After design of primers and probes we used a BLAST search to determine if there was cross-reactivity of the sequences to other organisms likely to be present at the site of collection. The primers and probes designed targeted DNA and were designed in a Taqman-MGB format.

1) 5.8S rRNA:

Alignments incorporated the 5.8S sequence from multiple TV strains with the following Genbank/EMBL accession numbers: L29561, AY871048, AY957955, AY871044, AY871046, AY871045, AY871047, U86613, AJ84785, AY245136, AY349186, AY349183, AY349185, AY349184. In addition, the 5.8S sequence from the following organisms were also included in the alignment: T. tenax (U86615, U37711), T. foetus (AF466751, U17509), Pentatrichomonas hominis (AF156964, AF342741, AY245137, AY 349187, AY 758392, U86616), T. gallinae (AY349182, U86614), T. canistomae (AY244652) and Trichomonas species from canis familiaris (AJ784785).

Primer Design

PCR primers were designed with a Tm of 58° C. to 60° C. and probes were designed with Tms that were 10° C. higher than the PCR primers (e.g., 68° C. to 70° C.). Primer pairs (forward and reverse primers) were designed to have similar Tms. Non-specific binding was minimized wherever possible by introducing instability at the 3′ end of the primer by keeping the number of G's and C's to about 2-3 in the last 5 bases at the 3′ end of the primers.

Probes were designed such that they did not start with a G residue, and designs with 3 adjacent G residues and those with a higher percentage of Gs than Cs were avoided.

Care was taken to balance the percent Gs and Cs in primers and probes as close as possible so that they would work together optimally. Primers were designed to be between 15 to 30 base pairs in length. Probes were designed to be between 20 to 30 base pairs in length. Amplicons were 50 to 150 by in length and designed such that the 5′ end of the probe was about 3 nucleotides from the 3′ end of the primer on the same strand. Candidate primer and probe sequences were also visually inspected for Tm, secondary structure, and complementarity using both Primer Express 3.0 and IDT OligoAnalyzer 3.0 so that there would be no bias introduced by any one analysis algorithm.

Multiple primer/probe pairs were designed as described above and then tested for specificity in silico using BLAST and using a reference strain of TV to identify promising detection reagents.

Based on the above criteria, the following primers and probes were chosen as candidates for TV_{—}5.8S rRNA PCR assay:

TABLE 1
Candidate primers and probe for TV 5.8 rRNA PCR
Assay

| | | | |
|---|---|---|---|
| | location | | |
| | on 5.8S | | |
| | (with | | |
| | ref. to | | SEQ |
| | SEQ ID | | ID |
| Reference | NO: 1) | Sequence | NO: |

Forward PCR Primers

| | | | |
|---|---|---|---|
| #134 | 119-138 | 5′-GCAATGGATGTCTTGGCTCC-3′ | 4 |
| (forward) | | | |
| #104/105 | 132-151 | 5′ TGGCTCCTCACACGATGAAG-3′ | 5 |
| (forward) | | | |
| #133 | 141-160 | 5′-ACACGATGAAGAACGTGGCA-3′ | 6 |
| (forward) | | | |
| #149 | 179-195 | 5′-CCGGAGTTGCAAACATCATG-3′ | 7 |
| (forward) | | | |
| #153 | 194-208 | 5′- | 8 |
| (forward) | | TGACAGGTTAATCTTTGAATGCA-3′ | |
| #155 | 198-210 | 5′- | 9 |
| (forward) | | AGGTTAATCTTTGAATGCAAATTGC- | |
| | | 3′ | |
| #156 | 224-242 | 5′-CTAAACTCGATCTCGGTCG-3′ | 10 |
| (forward) | | | |

Reverse PCR Primers

| | | | |
|---|---|---|---|
| #151 | 218-238 | 5′-CGAGATCGAGTTTAGCGCAAT- | 11 |
| (reverse) | | 3′ | |
| #154 | 212-232 | 5′-CGAGTTTAGCGCAATTTGCAT- | 12 |
| (reverse) | | 3′ | |
| #157 | 246-268 | 5′- | 13 |
| (reverse) | | GATGTAGTACTGTCACACCCATGCT- | |
| | | 3′ | |
| #106 | 251-279 | 5′- | 14 |
| (reverse) | | ATTATAAAAGATGTAGTACTGTCACA | |
| | | CCC-3′ | |
| #135 | 315-338 | 5′- | 15 |
| (reverse) | | GGCAGACTACGTGTTGTTTGTCTT- | |
| | | 3′ | |

Probes

| | | | |
|---|---|---|---|
| #150 | 197-216 | 5′-FAM- | 16 |
| (probe) | | CAGGTTAATCTTTGAATGCA-MGB- | |
| | | 3 | |

The candidate PCR primers were tested in an assay using the forward and reverse primers and the Taqman-MGB probe described in TABLE 1 to amplify and detect DNA from a portion of the 5.8S gene of Trichomonas vaginalis. Initial assay characterization was done on genomic template prepared from a laboratory strain, 30001D, purchased from ATCC. Subsequent analysis was done using strain PRA 98, also purchased from the ATCC.

Results:

For the 5.8S assay, several primers and probe combinations were tested with two strains of T. vaginalis (ATCC 30001D, 30247) and a strain of 7: Tenax (ATCC 30207). Cycle threshold (Ct) values were compared as a surrogate for the ultimate sensitivity of the assay, with a lower Ct value correlated with more robust detection and a higher Ct value correlated with less efficient detection. The primer pairs chosen for future assay development, as described below, detected the T. vaginalis strains at an average Ct value of 19.8 and T. Tenax at a Ct value of 26.7.

### EXAMPLE 2

This Example describes the use of Trichomonas vaginalis 28S rRNA Gene Target for a Diagnostic PCR Assay

### Rationale: 28S RRNA

A multiple sequence alignment done at very high stringency identified bases that were 100% conserved across all 28S sequences aligned (2431/2918 bases) and all assay design targeted these conserved regions. 28S was selected as a target based on sequence conservation and the fact that the gene is represented at equivalent copy numbers as the 5.8S rRNA gene also selected as a target in this project.

### Methods:

1. Assay Design

The design for the 28S rRNA assay utilized information from 210 TV 28S rRNA sequences which were aligned to determine conserved sequence regions that could be used to develop a PCR assay that could detect the presence of various strains of TV. The alignments of the 210 TV 28S rRNA sequences showed extremely little polymorphism across the length of TV 28S rRNA: 83% of bases are 100% conserved among all 210 contributing sequences. Consensus sequences were generated using SEAVIEW with the threshold set to 100% identity which excludes even one mismatch. Using this scaffold as a guide we designed a Taqman-MGB assay directed to conserved regions of the target.

In order to develop a PCR assay that would be specific for TV and not detect non-TV sequences, the design for the 28S rRNA assay also utilized information from 210 TV 28S rRNA sequences aligned to a consensus sequence of non TV 28S rRNA sequences by the Carlton laboratory and was in the form of alignments of consensus sequences from the TV genome sequencing project reported in Science 315:207-211 (2007).

The 28S rRNA sequence from a reference TV rRNA strain (Genbank Ref. No. AF202181 TV 28S rRNA) (SEQ ID NO:17) was aligned to the consensus sequence of non-T. vaginalis 28S rRNA sequence generated by Carlton et al.

Based on the above criteria, the following primers and probes were chosen as candidates for TV_{—}28S rRNA PCR assay:

TABLE 2
Candidate primers and probe for TV 28S rRNA
PCR Assay

| | location on | | |
|---|---|---|---|
| | 28S (with | | |
| | ref. to | | SEQ |
| | SEQ ID NO: | | ID |
| Reference | 16) | Sequence | NO: |
| #206 | 2653-2679 | 5′- | 20 |
| (forward) | | AGGTAACCCAATGTAGAAGACATTG | |
| | | TG-3′ | |
| #207 | 2721-2742 | 5′- | 21 |
| (reverse) | | GAAGCTGAACCTCAACAGGTCG-3′ | |
| #208 | 2688-2017 | 5′-6-FAM- | 22 |
| (probe) | | GGTACTGTAAGCAGTGGAG- | |
| | | MGBNFQ-3′ | |

Results:

For the 28S assay, the initial primer designs worked well. The primers and probe were tested with two strains of T. vaginalis (ATCC 30001D, 30247) and a strain of T. Tenax (ATCC 30207). The primers and probe detected all of the organisms at a concentration of 50 ng/reaction. T. Tenax came up at a cycle threshold (Ct) of 29 while strains of T. vaginalis came up at a Ct between 19.5 and 22.5. Fluorescence was acceptably high. With regard to distinguishing between T. vaginalis and T. Tenax in a test sample, it is noted that T. Tenax is not found in the same anatomical compartment as T. vaginalis.

Initial testing of this assay with reference T. vaginalis strains indicated that the assay was successful for both specific and sensitive detection of T. vaginalis, as described in Example 3.

### EXAMPLE 3

This Example describes the validation of Trichomonas vaginalis 5.8S and 28S rRNA Gene Target for use in a diagnostic PCR assay.

### Methods:

1. Testing of ATCC Reference Strains

Initial testing of the 5.8S assay and 28S assay was carried out to determine the assay performance with cultured and sequenced reference strains from ATCC using relatively large amounts of genomic DNA (50 ng/reaction). While these isolates were banked several years ago and therefore do not necessarily represent the strains present in human populations today, they are representative of the sequences on which our 5.8S PCR assay design is based.

Initial assay characterization using the PCR primers and probes described in Examples 1 and 2 was carried out on genomic template prepared from a laboratory strain, 30001D, purchased from ATCC, which was later found to be mis-classified. In this Example, the number of strains used as genomic templates was expanded to include other strains from ATCC: PRA-98, 30247, 50143, 50144 and 50145.

TABLE 3
Primers and Probe used for T. vaginalis 5.8S Assay

| | location on | | | | |
|---|---|---|---|---|---|
| Reference | 5.8S (with ref. to | | | | |
| Number | SEQ ID NO: 1) | SEQ ID | Length | Tm | % GC |
| #149 (F) | 179-195 | 7 | 17 nt | 59 | 50% |
| #151 (R) | 218-238 | 11 | 21 nt | 58 | 48% |
| #150 (probe) | 197-216 | 16 | 20 nt | 69 | 35% |

TABLE 4
Primers and Probe Used for T. vaginalis 28S Assay

| | location on 28S | | | | |
|---|---|---|---|---|---|
| | (with ref. to | SEQ ID | | | |
| Reference | SEQ ID NO: 17) | NO: | Length | Tm | % GC |
| #206 (forward | 2653-2679 | 20 | 27 nt | 56.8 | 40.7% |
| primer) | | | | | |
| #207 (reverse | 2721-2742 | 21 | 22 nt | 58.0 | 54.5% |
| primer) | | | | | |
| #208 (probe) | 2688-2017 | 22 | 19 nt | 52.2 | 52.6% |

PCR Methods for both 5.8S and 28S Assays:

Primers and Probes:

The primers were synthesized by ABI and delivered in lyophilized form. The primers were resuspended in nuclease-free water to a concentration of 100 pmol/μl which is equivalent to a concentration of 100 μM. This stock was kept at −20° C. and diluted 1:5 with nuclease-free water to give a working stock of 20 μM. The probe was kept at the 100 μM, undiluted concentration. All stocks were stored at −20° C. and thawed just prior to use.

DNA Sample Preparation

DNA was isolated using either manual extraction using Qiagen blood Mini kit (Cat #51104) from 200 μl of blinded specimens. Genomic DNA was eluted into 200 μl volume and 2.5 or 5 μl used as a template in a standard 25 μl PCR reaction. Assay conditions were standardized by using a PCR pre-mix such as ABI mastermix or Stratagene Brilliant II with ROX reference dye correction and by using the same annealing temperatures and cycling conditions

PCR Reactions:

TABLE 5
PCR Reaction Mix

| | Reagents per | Final concentration |
|---|---|---|
| Component | single reaction | per rxn |
| ABI 2X mastermix* | 12.5 μl | |
| Forward primer (20 μM) | 1.0 μl | 800 nM |
| Reverse primer (20 μM) | 1.0 μl | 800 nM |
| Probe (100 μM) | 0.025 μl | 200 nM |
| Template (10 ng gDNA) | 2.5 μl | |
| PCR grade H₂O | 7.975 μl | |
| Total volume | 25 μl | |

| Cycling parameters | | |
|---|---|---|
| Set Detector to FAM (no quench) | | |
| Initial 50° C., 2 min incubation for | | |
| UNG nuclease digestion | | |
| (optional step) | | |
| Initial denaturation at | 95° C., 10 mins | |
| | 40 cycles of | |
| | Denaturation | 95° C., 15 sec |
| | Anneal/Extension | 60° C., 1 min |

*(contains dNTPS, Taq, MgCl₂ and reaction buffer)

Data was collected during the 60° C. anneal/extension step.

TABLE 6
Performance of 5.8S and 28S rRNA assays using ATCC Reference Strains

| | | 5.8S assay | | 28S assay | |
|---|---|---|---|---|---|
| Sample | | Average | StDev | Average | StDev |
| ID | Type | Ct | Ct | Ct | Ct |
| PRA-98 | T. vaginalis type strain | 19.16 | 0.103 | 18.62 | 0.125 |
| | (10 ng) | | | | |
| 30247 | T. vaginalis type strain | 19.40 | 0.110 | 16.35 | 0.154 |
| | (10 ng) | | | | |
| 50143 | T. vaginalis type strain | 19.15 | 0.010 | 18.51 | 1.275 |
| | (10 ng) | | | | |
| 50144 | T. vaginalis type strain | 17.29 | 0.130 | 17.05 | 3.224 |
| | (10 ng) | | | | |
| 50145 | T. vaginalis type strain | 17.75 | 0.030 | 15.49 | 1.061 |
| | (10 ng) | | | | |
| 30001D | T. vaginalis type strain | 16.10 | 0.220 | 14.59 | 0.094 |
| | (10 ng) | | | | |
| 30207 | T. tenax (Near neighbor) | 26.99 | 0.050 | 28.92 | 0.243 |
| | strain (10 ng) | | | | |

Number of T. vaginalis strains tested: 6
Number of T. tenax strains tested: 1

2. Measuring Geographic Diversity and Estimating Sensitivity (UW Panels)

i) Set 1: 4, 813b, 4-2, 813B, 814B, 812b

To estimate the sensitivity of the assays using clinical materials two samples from geographically diverse sites in Peru were diluted and blinded. These samples were instances of samples that had been mis-handled with undocumented freeze-thaws and/or extended periods of room temperature storage. All of these samples had been originally tested positive at the time of original sample analysis using the GenProbe TMA assay (W. Whittington, archived data). As shown below in Table 7, both the 5.8S and 28S assays were able to detect all but one of these samples with the 28S assay giving a slightly lower Ct value correlating with a slightly earlier detection.

ii) Set 2: A1, A2, A3, A4

Four additional clinical samples from the same sample set collected in geographically diverse sites in Peru.

TABLE 7
Detection of geographically diverse clinical samples

| | | | 5.8S assay | | 28S assay | |
|---|---|---|---|---|---|---|
| Sample | | | Average | StDev | Average | StDev |
| ID | Type | Region | Ct | Ct | Ct | Ct |
| 4 | Peru | | 36.12 | 0.815 | Undet | n/a |
| 813b | Peru | | 33.63 | 0.105 | 34.71 | 0.713 |
| 4-2 | Peru | | Undet | n/a | Undet | n/a |
| 813B | Peru | | 36.08 | 0.548 | 36.39 | 0.872 |
| 814B | Peru | | 38.92 | 0.024 | 34.53 | 0.821 |
| 812b | Peru | | 35.25 | 0.248 | 36.50 | 1.460 |
| A1 | Peru | Highlands | 18.16 | 0.050 | 19.27 | 0.076 |
| A2 | Peru | Jungle | 23.37 | 0.061 | 23.37 | 0.0015 |
| A3 | Peru | Coastal | 20.74 | 0.013 | 20.74 | 0.0534 |
| A4 | Peru | Coastal | 21.89 | 0.112 | 23.00 | 0.028 |

Number samples tested = 10

3. Measuring Sensitivity Using Blinded Sensitivity Panels (UW Panels)

Sensitivity of the 5.8S and 28S rRNA assays was measured using various sets of templates as follows.

First, a few blinded dilutions of a quantitated clinical sample were tested in both assays (Sample IDs: NRL 1-3). The experiment utilized three concentrations of TV organisms (>10, 1-2, and <1 forms/high power field (hpf)) based on microscopic evaluation of culture specimens (InPouch, Biomed, White City, Oreg.) counted in four microscopic fields. In addition, 5.8S assays were performed in parallel with 5 and 10 ng of extracted material. Results summarized below in Table 3 are restricted to assays performed with 5 ng of extracted material.

Five further isolates (Sample IDs: 1-5 through 7-5) were from commercial sex workers seen in two Peruvian cities. Vaginal specimens were self-collected from several different regions of Peru, cultured in the laboratory in Peru and then preserved and shipped to a facility in Seattle. The specimens were grown in In-Pouch medium and passaged five times each before they were assayed. Numbers 4 and 7 were replicates; 1, 2, and 6 were sent once. The quantity of viable forms were estimated by counting the number of forms in pools of culture medium. All specimens yielded high quantities of trichomonads per specimen at each passage: 10-30 trichomonads per high powered field. Serial 10-fold dilutions were then prepared to estimate concentrations of organisms ranging from 10⁵ to 100 organisms/ml. 200 μl of each suspension was extracted and 5/200 μl of the extracted material tested in the 5.8S and 28S assays.

The results shown in TABLE 8 show sensitive detection down to a low number of organisms. One limitation in this estimate of sensitivity is the presence of non-viable organisms that while not contributing to the organism count are extracted from the culture along with the quantified parasite forms.

TABLE 8
Measuring Sensitivity Using blinded Sensitivity Panels

| | Quantity of | | No. of | 5.8S assay | | 28S assay | |
|---|---|---|---|---|---|---|---|
| Sample | organisms/ | | organisms/ | Average | StDev | Average | StDev |
| ISD | ml | | reaction | Ct | Ct | Ct | Ct |
| NRL-1 | >10 | forms/hpf | — | 21.8 | 0.15 | NT | |
| NRL-2 | 1-2 | forms/hpf | — | 26.3 | 0.11 | NT | |
| NRL-3 | <1 | form/hpf | — | 33.1 | 0.54 | NT | |

| | | | | | | |
|---|---|---|---|---|---|---|
| 1-5 | 10 | 0.05 | 25.34 | 0.056 | 22.61 | 0.224 |
| 6-5 | 100 | 0.5 | 24.88 | 0.2 | 21.3 | 0.02 |
| 4-5 | 1000 | 5 | 21.18 | 0.066 | 17.49 | 0.309 |
| 7-5 | 10000 | 50 | 20.42 | 0.04 | 18.4 | 0.485 |
| 2-5 | 100000 | 500 | 20.21 | 0.078 | 15.32 | 0.239 |

Number samples tested = 8

4. Measuring Sensitivity in Archived Vaginal Swab Specimens (Kurth Study)

Vaginal swabs were collected by clinicians as part of the “Xenotopc Study” (Kurth et al., J. Clin. Microbiol. 42:2940-3, 2004). At the time of the original study, swabs were placed in microfuge tubes containing 0.5 ml of sample buffer (nuclease-free phosphate-buffered saline [pH 7.4] containing 0.5% Triton X-100 and 0.01% NaN₃) within 18 hours of collection. The swabs were mixed for 1 min, and the solution expressed from the swab and used for testing by the Xenotope assay. The swab was then returned to a tube and stored at −70° C.

Specimen preparation for this study involved taking the archived swab, equilibrating it to room temperature and reconstituting with 1.5 ml of nuclease-free PBS (pH 7.4). The PBS and swab were mixed by vortexing for one minute and divided into three microfuge vials, yielding at least 400 microlitres in each vial. The resulting aliquots were then stored at −70° C. pending further analysis. One of the prepared vials was used for testing by the GenProbe TV-TMA assay (GenProbe Aptima ASR kit, product numbers 302078, 302080, 302077, 302079, 302076) and subsequently by the GenProbe Aptima Combo 2 assay (GenProbe, Product number 1032) to establish a standard concerning the pathogens of interest. The second vial was transported on dry ice, extracted using the Qiagen genomic miniprep kit and used to test the performance of both the 5.8S and 28S rRNA assays. The third vial was stored at −70° C. for possible future testing in modified assays or to resolve any apparent inconsistencies in initial results.

The total sample set size for this portion of the study was 192 specimens and the results from the two PCR assays were compared to both culture and GenProbe TMA assay results after assays were completed. Despite the fact that the GenProbe TMA assay is an RNA based assay, the 5.8S and 28S rRNA assays, targeting genomic DNA, matched favorably with the RNA results.

TABLE 9
Performance of 5.8S and 28S assay compared to GenProbe TMA assay

| | | | | | | Genprobe | | | | |
|---|---|---|---|---|---|---|---|---|---|---|
| | | | | Genprobe | Genprobe | Repeat | 5.8S assay | | 28S assay | |
| Sample | Culture | Culture 1 | Xenotope | Result 1 | Result 2 | FINAL | Avg | StDev | Avg | StDev |
| ID | Result | Day # | Result | (RLU) | (RLU) | (RLU) | Ct | Ct | Ct | Ct |
| 1 | 0 | | 0 | 1692 | | 1674 | | | | |
| 2 | 0 | | 0 | 1715 | | 1695 | | | | |
| 3 | 0 | | 0 | 1831 | | 1786 | | | | |
| 4 | 0 | | 0 | 1669 | | 1766 | | | | |
| 5 | 0 | | 0 | 1878 | | 1973 | 39.29 | 0.46 | 39.78 | |
| 6 | 1 | 1 | 1 | 4338290 | | 5691173 | | | | |
| 7 | 0 | | 0 | 2086 | | 1647 | 33.81 | 0.46 | 36.23 | 0.26 |
| 8 | 1 | 1 | 1 | 4689306 | 4415411 | 5497129 | 18.95 | 0.06 | 20.32 | 0.21 |
| 9 | 0 | | 0 | 1600 | | 1739 | | | | |
| 10 | 0 | | 0 | 1580 | | 1721 | 21.08 | 0.03 | 22.51 | 0.17 |
| 11 | 0 | | 0 | 1807 | | 1870 | | | | |
| 12 | 0 | | 0 | 1758 | | 1766 | | | | |
| 13 | 0 | | 0 | 1646 | | 1704 | 29.73 | 0.11 | 30.99 | 0.28 |
| 14 | 0 | | 0 | 1757 | | 1746 | | | | |
| 15 | 1 | 4 | 1 | 7702 | | 646220 | | | | |
| 16 | 0 | | 0 | 1666 | | 1716 | | | | |
| 17 | 0 | | 0 | 1779 | | 1724 | | | | |
| 18 | 0 | | 0 | 1734 | | 1747 | | | | |
| 19 | 1 | 4 | 1 | 91519 | 83638 | 1415695 | 21.09 | 0.12 | 22.59 | 0.16 |
| 20 | 0 | | 0 | 1698 | | 1778 | | | 34.99 | |
| 21 | 0 | | 0 | 1617 | | 1661 | | | | |
| 22 | 1 | 2 | 1 | 3922571 | | 5738172 | | | | |
| 23 | 0 | | 0 | 1644 | | 1729 | 39.64 | | | |
| 24 | 0 | | 0 | 1658 | | 1660 | 39.29 | | | |
| 25 | 0 | | 0 | 1667 | | 1720 | 38.55 | | | |
| 26 | 0 | | 0 | 24467 | 44007 | 393369 | 31.18 | 0.73 | 31.86 | 0.27 |
| 27 | 1 | 4 | 1 | 4535073 | 4473054 | 5547587 | 18.04 | 0.78 | 18.76 | 0.07 |
| 28 | 0 | | 0 | 1790 | | 1815 | 39.08 | | | |
| 29 | 0 | | 0 | 1762 | | 1845 | 39.83 | | | |
| 30 | 0 | | 0 | 1816 | | 1786 | | | | |
| 31 | 1 | 1 | 1 | 3537187 | | 4850622 | 20.78 | 0.86 | 21.74 | 0.50 |
| 32 | 0 | | 0 | 1649 | | 1801 | 38.18 | 0.77 | | |
| 33 | 0 | | 0 | 1769 | | 1762 | 39.52 | 0.08 | | |
| 34 | 0 | | 0 | 1713 | | 1747 | | | | |
| 35 | 1 | 1 | 1 | 573738 | 676263 | 4182992 | 24.58 | 0.88 | 25.23 | 0.12 |
| 36 | 0 | | 0 | 1737 | | 1925 | | | | |
| 37 | 0 | | 0 | 1557 | | 1688 | 39.00 | 0.55 | | |
| 38 | 0 | | 0 | 1673 | | 1749 | 39.59 | 0.30 | | |
| 39 | 0 | | 0 | 1821 | | 1692 | 39.22 | | | |
| 40 | 1 | 2 | 0 | 2747549 | 3266761 | 5327911 | 29.86 | 0.77 | 30.53 | 0.01 |
| 41 | 0 | | 0 | 1735 | | 1870 | 39.01 | | | |
| 42 | 0 | | 0 | 1641 | | 1681 | 39.31 | | | |
| 43 | 1 | 1 | 1 | 2545400 | 2507399 | 5410834 | 25.14 | 0.88 | 25.80 | 0.08 |
| 44 | 0 | | 0 | 1605 | | 1730 | | | | |
| 45 | 0 | | 0 | 1608 | | 1705 | | | | |
| 46 | 1 | 1 | 1 | 4139250 | 4150079 | 5646763 | 25.58 | 0.73 | 25.75 | 0.02 |
| 47 | 0 | | 0 | 1590 | | 1672 | | | | |
| 48 | 0 | | 0 | 1620 | | 1632 | 37.39 | 2.26 | | |
| 49 | 1 | 1 | 1 | 71425 | 96436 | 917392 | 26.18 | 0.03 | 29.34 | 0.43 |
| 50 | 0 | | 0 | 1630 | | 1683 | 37.39 | | | |
| 51 | 0 | | 0 | 1773 | | 1771 | 39.73 | 0.26 | | |
| 52 | 0 | | 0 | 1822 | | 1895 | 37.40 | | | |
| 53 | 0 | | 0 | 1758 | | 1736 | | | | |
| 54 | 0 | | 0 | 1637 | | 1723 | 38.69 | | | |
| 55 | 0 | | 0 | 1720 | | 1804 | | | | |
| 56 | 1 | 1 | 1 | 2386646 | | 4264843 | 22.64 | 0.03 | 25.83 | 0.46 |
| 57 | 0 | | 0 | 1692 | | 1695 | 39.14 | | | |
| 58 | 0 | | 0 | 1741 | | 1658 | | | | |
| 59 | 0 | | 0 | 1905 | | 2033 | 39.25 | | | |
| 60 | 1 | 1 | 1 | 3492135 | 3595156 | 5431216 | 20.36 | 0.11 | 23.19 | 0.42 |
| 61 | 0 | | 0 | 1745 | | 1842 | 39.47 | | | |
| 62 | 1 | 1 | 1 | 4382883 | 4446490 | 5483015 | 20.38 | 0.16 | 23.46 | 0.43 |
| 63 | 0 | | 0 | 1649 | | 1723 | 38.89 | 0.06 | | |
| 64 | 0 | | 0 | 1666 | | 1768 | | | | |
| 65 | 0 | | 0 | 1640 | | 1829 | 38.89 | | | |
| 66 | 1 | 1 | 1 | 4230456 | 4165594 | 5508826 | 20.39 | 0.06 | 23.71 | 0.40 |
| 67 | 0 | | 0 | 1674 | | 1677 | 36.89 | | | |
| 68 | 0 | | 0 | 1755 | | 1755 | 38.65 | | | |
| 69 | 0 | | 0 | 1670 | | 1692 | 37.15 | | | |
| 70 | 0 | | 0 | 1532 | 1510 | 1651 | 37.56 | 0.79 | | |
| 71 | 1 | 6 | 0 | 6303 | 3619 | 1743 | 31.24 | 0.09 | 34.20 | 0.68 |
| 72 | 0 | | 0 | 1613 | | 1737 | 38.14 | 1.52 | | |
| 73 | 0 | | 0 | 1695 | | 1750 | | | | |
| 74 | 0 | | 0 | 1527 | | 1654 | | | | |
| 75 | 1 | 2 | 1 | 1737950 | | 3853948 | 26.62 | 0.08 | 30.30 | 0.12 |
| 76 | 0 | | 0 | 1645 | | 1656 | 38.27 | 0.01 | | |
| 77 | 0 | | 0 | 1573 | | 1696 | | | | |
| 78 | 1 | 5 | 0 | 853466 | 500379 | 5232900 | 29.00 | 0.03 | 32.14 | 0.16 |
| 79 | 0 | | 0 | 1604 | | 1771 | | | | |
| 80 | 0 | | 0 | 1632 | | 1866 | | | | |
| 81 | 0 | | 0 | 1493 | | 1855 | 35.84 | | | |
| 82 | 1 | 1 | 1 | 4379862 | | 5572356 | 19.39 | 0.07 | 22.59 | 0.08 |
| 83 | 0 | | 0 | 1681 | | 1699 | 39.08 | 0.03 | | |
| 84 | 1 | 1 | 1 | 4264781 | | 5308471 | 18.65 | 0.10 | 22.11 | 0.02 |
| 85 | 1 | 4 | 0 | 8690 | 10868 | 1763 | 34.30 | 0.09 | 38.28 | 1.24 |
| 86 | 0 | | 0 | 1514 | | 1582 | | | | |
| 87 | 0 | | 0 | 1554 | | 1654 | | | | |
| 88 | 1 | 1 | 1 | 102596 | 71083 | 4364948 | 23.12 | 0.02 | 26.62 | 0.17 |
| 89 | 0 | | 0 | 1583 | | 1665 | 39.19 | | | |
| 90 | 0 | | 0 | 1479 | | 1765 | | | | |
| 91 | 0 | | 0 | 1413 | | 1808 | | | | |
| 92 | 1 | 2 | 1 | 3967748 | | 5592700 | 21.18 | 0.13 | 24.63 | 0.02 |
| 93 | 0 | | 0 | 1468 | | 1740 | | | | |
| 94 | 0 | | 0 | 1453 | | 1736 | 38.41 | 0.53 | | |
| 95 | 1 | 1 | 1 | 4189805 | | 5560143 | 20.10 | 0.04 | 23.41 | 0.05 |
| 96 | 0 | | 0 | 1506 | | 1629 | 17.26 | 0.09 | | |
| 97 | 0 | | 0 | | | 1909 | | | | |
| 98 | 0 | | 0 | | | 1866 | | | | |
| 99 | 0 | | 0 | | | 1947 | | | | |
| 100 | 1 | 1 | 1 | | | 5538340 | 20.40 | 0.02 | 22.70 | 0.32 |
| 101 | 0 | | 0 | | | 2031 | | | | |
| 102 | 0 | | 0 | | | 1923 | | | | |
| 103 | 0 | | 0 | | | 1945 | | | | |
| 104 | 0 | | 0 | | | 1854 | | | | |
| 105 | 0 | | 0 | | | 1883 | | | | |
| 106 | 0 | | 0 | | | 1804 | | | | |
| 107 | 1 | 1 | 1 | | | 1440341 | 29.21 | 0.06 | 31.45 | 0.43 |
| 108 | 0 | | 0 | | | 1979 | | | | |
| 109 | 0 | | 0 | | | 1885 | | | | |
| 110 | 0 | | 0 | | | 1841 | | | | |
| 111 | 0 | | 0 | | | 1874 | | | | |
| 112 | 0 | | 0 | | | 1941 | | | | |
| 113 | 1 | 1 | 1 | | | 5002214 | 24.62 | 0.26 | 27.34 | 0.38 |
| 114 | 0 | | 0 | | | 2046 | | | | |
| 115 | 0 | | 0 | | | 2033 | | | | |
| 116 | 0 | | 0 | | | 1957 | | | | |
| 117 | 0 | | 0 | | | 1908 | | | | |
| 118 | 0 | | 0 | | | 1875 | | | | |
| 119 | 0 | | 0 | | | 1836 | | | | |
| 120 | 0 | | 0 | | | 1890 | | | | |
| 121 | 1 | 1 | 1 | | | 5368686 | 25.00 | 0.74 | 26.79 | 0.78 |
| 122 | 0 | | 0 | | | 2007 | | | | |
| 123 | 0 | | 0 | | | 1902 | | | | |
| 124 | 0 | | 0 | | | 1816 | | | | |
| 125 | 0 | | 0 | | | 1816 | | | | |
| 126 | 0 | | 0 | | | 1771 | | | | |
| 127 | 0 | | 0 | | | 1782 | | | | |
| 128 | 0 | | 0 | | | 1817 | | | | |
| 129 | 1 | 1 | 1 | | | 4268456 | 20.70 | 0.20 | 23.08 | 0.91 |
| 130 | 0 | | 0 | | | 1839 | | | | |
| 131 | 0 | | 0 | | | 1971 | | | | |
| 132 | 0 | | 0 | | | 2181 | | | | |
| 133 | 0 | | 0 | | | 1882 | | | | |
| 134 | 0 | | 0 | | | 1963 | | | | |
| 135 | 1 | 1 | 1 | | | 3933063 | 26.87 | 0.45 | 29.17 | 0.45 |
| 136 | 0 | | 0 | | | 2059 | | | | |
| 137 | 0 | | 0 | | | 1921 | | | | |
| 138 | 0 | | 0 | | | 1884 | | | | |
| 139 | 0 | | 0 | | | 1853 | | | | |
| 140 | 0 | | 0 | | | 1839 | | | | |
| 141 | 0 | | 0 | | | 1795 | | | | |
| 142 | 0 | | 0 | | | 1856 | | | | |
| 143 | 0 | | 0 | | | 1869 | | | | |
| 144 | 1 | 1 | 1 | | | 4768164 | 25.23 | 0.27 | 26.60 | 0.14 |
| 145 | 0 | | 0 | | | 1813 | | | | |
| 146 | 0 | | 0 | | | 1822 | | | | |
| 147 | 0 | | 0 | | | 1850 | | | | |
| 148 | 1 | 1 | 1 | | | 5656324 | 18.63 | 0.21 | 19.87 | 0.19 |
| 149 | 0 | | 0 | | | 1955 | | | | |
| 150 | 0 | | 0 | | | 1938 | | | | |
| 151 | 0 | | 0 | | | 1939 | | | | |
| 152 | 0 | | 0 | | | 1941 | | | | |
| 153 | 0 | | 0 | | | 1880 | | | | |
| 154 | 0 | | 0 | | | 1915 | | | | |
| 155 | 0 | | 0 | | | 1903 | | | | |
| 156 | 0 | | 0 | | | 1951 | | | | |
| 157 | 0 | | 0 | | | 18803 | 35.20 | 1.87 | 35.81 | 0.82 |
| 158 | 0 | | 0 | | | 1776 | | | | |
| 159 | 0 | | 0 | | | 1729 | | | | |
| 160 | 1 | 1 | 1 | | | 5480452 | 17.44 | 0.69 | 18.14 | 0.06 |
| 161 | 0 | | 0 | | | 1862 | | | | |
| 162 | 0 | | 0 | | | 1842 | | | | |
| 163 | 0 | | 0 | | | 1930 | | | | |
| 164 | 0 | | 0 | | | 1964 | | | | |
| 165 | 0 | | 0 | | | 1883 | | | | |
| 166 | 0 | | 0 | | | 1809 | | | | |
| 167 | 1 | 3 | 0 | | | 1989349 | 27.59 | 0.57 | 28.92 | 0.14 |
| 168 | 0 | | 0 | | | 18361 | | | | |
| 169 | 0 | | 0 | | | 1865 | | | | |
| 170 | 0 | | 0 | | | 1844 | | | | |
| 171 | 0 | | 0 | | | 1815 | | | | |
| 172 | 0 | | 0 | | | 1813 | | | | |
| 173 | 0 | | 0 | | | 1817 | | | | |
| 174 | 0 | | 0 | | | 1833 | | | | |
| 175 | 1 | 2 | 1 | | | 5488718 | 21.84 | 0.27 | 22.50 | 0.43 |
| 176 | 0 | | 0 | | | 1820 | | | | |
| 177 | 0 | | 0 | | | 1892 | | | | |
| 178 | 0 | | 0 | | | 1159601 | 28.21 | 0.15 | 29.23 | 0.55 |
| 179 | 0 | | 0 | | | 1839 | | | | |
| 180 | 0 | | 0 | | | 1821 | | | | |
| 181 | 1 | 2 | 0 | | | 2528773 | 27.60 | 0.06 | 29.43 | 0.42 |
| 182 | 0 | | 0 | | | 1808 | | | | |
| 183 | 0 | | 0 | | | 1770 | | | | |
| 184 | 0 | | 0 | | | 1842 | | | | |
| 185 | 0 | | 0 | | | 1860 | | | | |
| 186 | 0 | | 0 | | | 2064 | | | | |
| 187 | 0 | | 0 | | | 1913 | | | | |
| 188 | 0 | | 0 | | | 1804 | | | | |
| 189 | 1 | 1 | 1 | | | 5374565 | 27.16 | 0.07 | 27.40 | 0.08 |
| 190 | 0 | | 0 | | | 1770 | | | | |
| 191 | 0 | | 0 | | | 1783 | | | | |
| 192 | 0 | | 0 | | | 1820 | | | | |

(1 = growth: 0 = no growth in column 2)
Number samples tested = 192

Correlation analysis of GenProbe TMA assay with the 5.8S rRNA and 28S rRNA TV assays (for the studies described above) shows that the two assays are highly correlated as is shown in Tables 10 and 11 (following)

TABLE 10
5.8S rRNA assay

Result 5.8S

| | Neg | Pos | |
|---|---|---|---|
| TV-TMA Negative | 82 | 0 | 82 |
| Positive | 0 | 14 | 14 |
| Total | 82 | 14 | 96 |

TABLE 11
28S rRNA assay

Result 28S

| | Neg | Pos | |
|---|---|---|---|
| TV-TMA Negative | 82 | 0 | 82 |
| Positive | 0 | 14 | 14 |
| Total | 82 | 14 | 96 |

For each assay, a difference of 5 cycles between the mean value and the value defining an undetectable or negative result (Ct=40) was used to define a positive. This translated to a cut-off Ct value of 35 to define a positive result. Agreement with GenProbe TMA assay was 100% for each assay.

### EXAMPLE 4

This Example describe the use of the assays in 119 blinded samples collected from several STD clinics in New York City.

Methods:

119 blinded samples were received from the Carlton lab in two formats: extracted DNA and 1 frozen stabilate of each. The samples were collected from several STD clinics in New York City after the swabs were used for standard STD screening by smears, grown in InPouch for 5-7 days. An aliquot was frozen and the remainder DNA extracted for each sample. The minimal handling of these clinical specimens made them very close to the ‘point-of-origin’. The extracted genomic DNA received was tested in both the 5.8S rRNA and 28S rRNA assays. All the positives sent were correctly identified by the assays but the assays also identified 13 as being positive whereas our InPouch culture and diagnostic PCR methods were negative. Re-culturing of false positive samples and blinding of specimens resulted in re-calculating the cut-off values for both assays. Re-setting the cut-off for a positive sample to a Ct of 35 resulted in a resolution of the majority of the problem of false positives and yielded data that was more consistent with the NYU re-culture results. There were still discrepancies with culture negative samples that gave Ct values between 32 and 35. This could be due to the relative efficiencies and sensitivities of quantitative PCR methods when compared to In Pouch culture.

TABLE 12
Performance of 5.8S and 28S rRNA assays with culture
confirmed isolates from NYU

| | | 5.8S assay | | 28S assay | |
|---|---|---|---|---|---|
| | | | StDev | Average | |
| Sample ID | Culture Result | Average Ct | Ct | Ct | StDev Ct |
| BUSH01 | Neg | 33.73 | 0.42 | 33.60 | 0.65 |
| BUSH04 | Pos* | 19.13 | 0.13 | 19.33 | 0.17 |
| BUSH09 | Neg | 36.71 | 0.40 | 36.78 | 0.56 |
| BUSH13 | Neg | 38.20 | 1.47 | 38.07 | 1.28 |
| BUSH20 | Pos* | 21.70 | 0.16 | 21.72 | 0.30 |
| BUSH23 | Neg | 39.07 | | 38.72 | |
| CHAR01 | Neg | 38.58 | 0.99 | | |
| CHAR02 | Neg | | | | |
| CHAR03 | Neg | | | | |
| CHAR04 | Neg | 39.49 | | | |
| CHAR05 | Neg | | | | |
| CHAR09 | Neg | 35.75 | 0.57 | 35.52 | 0.42 |
| CHAR11 | Neg | 33.97 | 0.42 | 34.32 | 0.56 |
| CHAR12 | Neg | 35.07 | 0.16 | 35.48 | 0.22 |
| CHAR13 | Neg | 36.42 | 0.69 | 36.22 | 1.26 |
| CHAR15 | Neg | 36.27 | 0.64 | 35.93 | 0.41 |
| CHAR17 | Neg | 33.90 | 0.20 | 33.94 | 0.44 |
| CHAR18 | Neg | 36.36 | 0.52 | 36.77 | 1.02 |
| CHAR19 | Neg | 36.26 | 0.41 | 36.48 | 0.86 |
| CHAR20 | Neg | 34.16 | 0.26 | 34.00 | 0.76 |
| CHAR22 | Neg | 37.44 | 0.76 | 37.15 | 0.70 |
| CHAR24 | Neg | 37.16 | 1.48 | 36.93 | 1.25 |
| CHAR25 | Pos* | 22.23 | 0.11 | 22.13 | 0.60 |
| CHAR27 | Neg | 35.69 | 0.71 | 36.16 | 0.86 |
| CHAR28 | Neg | 37.95 | 0.71 | 37.88 | 0.55 |
| CHAR29 | Pos* | 21.10 | 0.03 | 21.64 | 0.53 |
| CHAR30 | Pos* | 21.09 | 0.17 | 21.41 | 0.70 |
| CHAR31 | Neg | 38.23 | 0.87 | 37.25 | 0.94 |
| CHAR32 | Neg | 37.08 | 1.11 | 38.44 | 1.21 |
| CHAR33 | Neg | 38.54 | 0.65 | 38.85 | 0.69 |
| CHAR35 | Neg | 36.10 | 1.33 | 36.31 | 0.80 |
| CHAR36 | Neg | 36.97 | 1.16 | 37.05 | 0.35 |
| CHAR37 | Pos* | 20.90 | 0.24 | 21.30 | 0.60 |
| CHAR38 | Neg | 34.82 | 0.31 | 35.15 | 0.09 |
| CHEL02 | Neg | 32.70 | 0.27 | 32.91 | 0.50 |
| CHEL04 | Neg | 34.41 | 0.41 | 34.69 | 0.30 |
| CHEL05 | Neg | 36.99 | 0.79 | 36.48 | 0.35 |
| CHEL06 | Neg | 34.97 | 0.76 | 34.86 | 0.08 |
| CHEL07 | Neg | 37.30 | 0.79 | 38.49 | 0.26 |
| CHEL08 | Neg | 37.55 | 0.95 | 38.36 | 1.62 |
| CHEL10 | Neg | 35.57 | 0.40 | 35.14 | 0.33 |
| CHEL11 | Neg | 36.71 | 1.81 | 36.16 | 0.57 |
| CHEL12 | Neg | 33.82 | 0.61 | 35.07 | 1.57 |
| CHEL13 | Neg | 35.53 | 0.98 | 34.27 | 0.69 |
| CHEL14 | Neg | 38.21 | 1.69 | 37.65 | 0.89 |
| CHEL15 | Pos* | 20.21 | 0.08 | 20.93 | 0.16 |
| CHEL17 | Neg | 37.93 | 0.01 | 39.76 | 0.03 |
| CHEL18 | Neg | 38.15 | 0.76 | 38.62 | 0.92 |
| CHEL19 | Neg | 38.03 | 1.32 | 38.96 | 0.72 |
| CHEL23 | Pos* | 18.66 | 0.07 | 19.69 | 0.17 |
| CHEL26 | Neg | 35.71 | 0.85 | 36.53 | 1.00 |
| CHEL27 | Neg | 32.76 | 0.60 | 34.65 | 1.16 |
| CHEL29 | Neg | 36.32 | 0.76 | 37.71 | 0.41 |
| CHEL31 | Neg | 37.43 | 0.92 | 38.28 | 1.00 |
| CHEL38 | Neg | 38.73 | 1.22 | 39.08 | |
| CHEL40 | Neg | 37.79 | 1.53 | 38.67 | 0.73 |
| CHEL41 | Neg | 36.42 | 0.49 | 37.66 | 0.67 |
| COR01 | Neg | 39.60 | | | |
| COR02 | Neg | 39.12 | 0.12 | | |
| COR03 | Neg | 34.00 | 0.35 | 34.71 | 0.54 |
| COR04 | Neg | 31.19 | 0.05 | 32.56 | 0.31 |
| COR10 | Neg | 37.27 | 1.09 | 37.66 | 0.38 |
| COR13 | Neg | 35.74 | 0.47 | 37.12 | 0.82 |
| COR16 | Neg | 37.66 | 0.64 | 38.62 | 0.15 |
| COR18 | Neg | 35.89 | 0.59 | 37.66 | 0.63 |
| COR19 | Neg | 36.50 | 0.49 | 38.00 | 0.88 |
| COR21 | Neg | 39.53 | 0.25 | | |
| COR22 | Neg | | | | |
| COR23 | Neg | 38.91 | 0.08 | | |
| COR24 | Neg | 38.54 | | | |
| COR30 | Neg | 34.71 | 0.80 | 36.29 | 0.52 |
| COR32 | Neg | 37.06 | 0.17 | 38.71 | 0.40 |
| COR35 | Neg | 35.35 | 0.74 | 36.68 | 0.64 |
| FTG02 | Pos* | 19.81 | 0.53 | 21.24 | 0.08 |
| FTG03 | Pos* | 18.76 | 0.17 | 19.62 | 0.67 |
| FTG04 | Neg | 36.82 | 0.75 | 38.42 | 0.65 |
| FTG10 | Neg | 37.92 | 0.58 | 38.56 | |
| FTG13 | Neg | 38.25 | 1.14 | | |
| FTG15 | Neg | 38.62 | 0.83 | | |
| FTG16 | Pos* | 19.03 | 0.10 | 20.09 | 0.12 |
| FTG17 | Neg | 37.77 | 0.81 | 38.48 | 0.61 |
| FTG20 | Neg | 36.45 | 0.77 | 33.88 | 4.65 |
| FTG31 | Neg | 34.67 | 0.51 | 35.93 | 0.31 |
| FTG32 | Pos* | 19.61 | 0.23 | 20.36 | 0.25 |
| FTG33 | Pos* | 18.76 | 0.24 | 19.42 | 0.30 |
| FTG36 | Neg | 36.18 | 0.84 | 37.76 | 1.24 |
| FTG41 | Neg | 37.57 | | 38.80 | |
| FTG42 | Neg | 38.01 | 0.23 | 39.06 | 0.80 |
| FTG45 | Neg | 38.03 | 0.25 | | |
| FTG46 | Neg | | | | |
| FTG47 | Neg | | | | |
| JAM13 | Pos* | 19.88 | 0.23 | 21.09 | 0.23 |
| JAM20 | Pos* | 15.92 | 2.09 | 19.50 | 0.06 |
| JAM28 | Pos* | 14.27 | 0.06 | 17.73 | 0.10 |
| MOR01 | Pos* | 17.79 | 0.41 | 20.36 | 0.14 |
| MOR06 | Neg | | | | |
| MOR07 | Neg | | | | |
| MOR13 | Neg | 35.34 | 0.14 | 37.96 | 0.22 |
| MOR14 | Neg | 34.40 | | 38.16 | |
| MOR19 | Pos* | 19.22 | 0.02 | 21.67 | 0.22 |
| MOR22 | Neg | 37.24 | | 29.33 | |
| MOR23 | Neg | 37.69 | 0.24 | | |
| MOR30 | Neg | 37.25 | | | |
| MOR31 | Pos* | 19.09 | 0.11 | 21.35 | 0.19 |
| MOR32 | Neg | 36.21 | 0.05 | 38.73 | 0.34 |
| MOR33 | Neg | 38.29 | | | |
| MOR34 | Neg | 37.55 | 0.56 | 39.74 | 0.17 |
| RIV01 | Neg | 32.69 | 1.10 | 35.03 | 0.23 |
| RIV02 | Neg | 32.69 | 0.72 | 35.21 | 0.15 |
| RIV04 | Neg | 31.09 | 0.13 | 33.98 | 0.33 |
| RIV05 | Neg | 34.79 | 0.27 | 37.40 | 0.40 |
| RIV06 | Neg | 37.79 | 1.07 | 39.13 | 0.92 |
| RIV15 | Neg | 36.69 | 0.55 | | |
| RIV16 | Neg | 36.39 | | | |
| RIV20 | Neg | 38.54 | | 38.92 | |
| RIV21 | Neg | 34.23 | 1.04 | 38.26 | 0.13 |
| RIV22 | Neg | 36.90 | 2.36 | 39.95 | |
| RIV27 | Neg | 35.61 | 0.52 | 37.72 | 0.41 |
| RIV28 | Neg | 36.11 | 1.16 | 38.69 | 0.64 |

| | |
|---|---|
| Number samples tested = | 117 |

*indicates a result with high confidence due to the fact it that has a low Ct value and a culture positive result. The “gold standard” test used with these samples was culture. TMA testing was not done with these samples because they were collected at the New York City STD clinic under conditions that did not preserve the RNA required for GenProbe TMA testing.
Note:
Ct values in the range of over 35, such as 35-38 represent clinical samples with none or extremely few organisms and are due to non-specific or false positive PCR results.

### CONCLUSIONS

The 5.8S and 28S rRNA DNA-based assays work robustly to detect reference, clinical, and cultured TV specimens from diverse geographical locations. The results correlate favorably to the gold standard RNA-based reference test, the GenProbe Aptima Combo 2, and with culture results at Ct values <30. The 5.8S and 28S rRNA DNA-based assays described herein were further validated in a set of 400 clinical samples from commercial sex workers from Mombasa, Kenya. Test results were compared to the GenProbe APTIMA TV assay and to cultures and showed excellent concordance (data not shown).

As will be appreciated by those of skill in the art, a highly sensitive DNA-based test provides significant advantages over an RNA-based test. For example, an RNA-based test requires careful handling of the sample to avoid RNA degradation. In addition to not requiring special handling, such as RNA stabilization buffers and ultra-clean plastics typically not found in clinic based settings, a DNA sample, stabilized or not, is more robust and resistant to mis-handling and can be stored at ambient temperatures longer than a corresponding RNA sample. In addition, a DNA sample provides a template that can be amplified directly from an extracted specimen, while an RNA sample requires extraction, reverse transcription and amplification. Although there are enzyme mixes that can do the reverse transcription and amplification simultaneously, these reagents are significantly more costly than those for DNA amplification only. Due to the high degree of sequence homology in the 5.8S gene between Trichomonas vaginalis and Trichomonas tenax, the assay will also detect DNA from T. tenax (although at higher Ct values). However, it is noted that T. Tenax is not found in the same anatomical compartment as T. vaginalis, therefore assay specificity for T. vaginalis versus T. Tenax is not likely to be an issue for clinical test samples obtained from vaginal and cervical swabs.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for determining the presence of Trichomonas vaginalis (TV) in a test sample, said method comprising the steps of:
(a) contacting a test sample with a composition comprising at least one primer pair comprising a forward and reverse primer capable of hybridizing to a target region of TV 28S consisting of SEQ ID NO:1.8 to form a reaction mixture; and
(b) subjecting said reaction mixture to amplification conditions suitable to amplify at least a portion of said target region.

2. The method of claim 1, wherein the composition comprises at least one primer pair comprising a forward and reverse primer capable of hybridizing to a target region of 28S consisting of SEQ ID NO:19.

3. The method of claim 1, further comprising detecting the presence of the amplified portion by contacting the reaction mixture with a detection probe under hybridizing conditions, wherein the detection probe has a nucleotide sequence that hybridizes to at least a portion of the amplified target region and determining the presence of a hybrid.

4. The method of claim 1, wherein the composition comprises a primer having a target binding region consisting of SEQ ID NO:20.

5. The method of claim 1, wherein the composition comprises a primer having a target binding region consisting of SEQ ID NO:21.

6. The method of claim 3, wherein the detection probe comprises a target binding region consisting of SEQ ID NO:22.

7. The method of claim 1, wherein determining the presence of said hybrid in said reaction mixture indicates the presence of TV in said test sample.

8. The method of claim 7, wherein determining the presence of said hybrid in said reaction mixture indicates the presence of TV in said test sample and the absence of T. tenax in said test sample.

9. A set of oligonucleotides for use in amplifying a target region of nucleic acid derived from 2S Trichomonas vaginalis, the set of oligonucleotides comprising a forward and reverse primer, each primer having a target binding region up to 30 nucleotides in length which contains at least 10 contiguous nucleotides which are perfectly complementary to an at least 10 contiguous nucleotide region present in a target sequence consisting of SEQ ID NO:19.

10. The set of oligonucleotides of claim 9, wherein the forward primer consists of SEQ ID NO:20.

11. The set of oligonucleotides of claim 9, wherein the reverse primer consists of SEQ ID NO:21.

12. The set of oligonucleotides of claim 9, further comprising a detection probe consisting of SEQ ID NO:22.

13. An oligonucleotide for use in amplifying a target region of nucleic acid derived from Trichomona vaginalis, said oligonucleotide having a target binding region of up to 30 bases in length which stably hybridizes to a target sequence selected from the group consisting of SEQ ID NO: 18 and SEQ ID NO:19.

14. The oligonucleotide of claim 13, wherein said target binding region contains at least 10 contiguous nucleotides that are perfectly complementary to at least 10 contiguous nucleotides in said target sequence.

15. The oligonucleotide of claim 13, wherein said oligonucleotide does not stably hybridize to SEQ ID NO:23, a nucleic acid sequence derived from T. tenax.

16. The oligonucleotide of claim 13, wherein said target region consists of SEQ ID NO:19.

17. A kit for determining the presence of Trichomonas vaginalis (TV) in a test sample, the kit comprising:
(a) at least one oligonucleotide comprising a target binding region sequence selected from the group consisting of SEQ ID NO:20, SEQ ID NO:21 and SEQ ID NO:22;
(b) amplification reagents; and
(c) written instructions describing amplification conditions suitable to distinguish between the presence of TV and Trichomonas tenax in the test sample.

18. The kit of claim 17, wherein one or more of the oligonucleotides incorporates one or more detectable labels.
